(19) United States
(12) Patent Application Publication
(10) Pub. No.: US 2024/0043391 A1
(43) Pub. Date: Feb. 8, 2024

(54) METHOD FOR PRODUCING ALKYLPYRAZINES

(71) Applicant: SYMRISE AG, Holzminden (DE)

(72) Inventors: Jekaterina ONGOUTA, Stadtoldendorf (DE); Michael BACKES, Holzminden (DE); Nadine HEINEMEIR, Bevern (DE); Thomas RIESS, Holzminden (DE); Volkmar KOPPE, Höxter-Stahle (DE); Stefan BRANDT, Vahlbruch (DE); Tobias VÖSSING, Beverungen (DE); Carina ROSE, Holzminden (DE); Dietmar SCHATKOWSKI, Einbeck (DE)

(21) Appl. No.: 18/263,727

(22) PCT Filed: Feb. 2, 2021
(86) PCT No.: PCT/EP2021/052408

§ 371 Date: Aug. 1, 2023

(51) Int. Cl. C07D 241/12

(52) U.S. Cl. CPC C07D 241/12

(57) ABSTRACT

The present invention primarily relates to a process for producing alkylpyrazines, the process comprising thermal treatment of an amino acid source in a high boiling solvent, the amino acid source containing at least threonine and/or serine and the thermal treatment yielding alkylpyrazines; and separation of the alkylpyrazines from the high boiling solvent.

The present invention primarily relates to a process for producing alkylsubstituted pyrazines (alkylpyrazines) and their use as an aroma and flavoring substance.

### BACKGROUND OF THE INVENTION

Alkylsubstituted pyrazines are valuable aroma compounds. Because of their typical roasted, nutty, chocolate flavor and taste alkylpyrazines are widely used for the modification and improvement of flavor compositions (FR 1530436 A1; U.S. Pat. No. 3,579,353 A, FR 2128744 A5, U.S. Pat. No. 3,924,015 A, JP 11313635 A, JP 2015044895 A, JP 2018130057 A). Alkylsubstituted pyridines were also found to be important aroma compounds with a typical roasted flavor (U.S. Pat. No. 4,005,227 A, JP 2000139397 A, JP 2005015683 A).

Pyrazines can be found ubiquitously in nature but only in relatively low amounts (0.001 and ppm; Applied microbiology and biotechnology, 2010, 85, 1315-1320). Pyrazines are produced naturally by living organisms including plants, animals, insects and marine organisms as well as microorganism (Expert Opinion on Therapeutic Patents 2015, 25, 33-47). In addition, alkylsubstituted pyrazines can be found in heat-processed foods such as coffee, cocoa, nuts, meat, cereals, rice and spices as they are formed through Maillard reaction. The formation of alkylpyridines via Maillard reaction has also been described in several cases (Journal of Chromatographic Science 1996, 34, 213-218). The Maillard reaction is a non-enzymatic browning of food that involves condensation of a carbonyl compound and an amine, which are a carbon source in the form of a reducing sugar and an amino acid, a peptide or a protein, respectively (Nursten, H. E. 2005. The Maillard reaction: chemistry, biochemistry, and implications. London, UK: Royal Society of Chemistry).

To date, a large number of studies has been carried out on pyrazine formation in sugar-amino acid systems. Different sugars such as fructose, glucose, saccharose, ribose, maltose, rhamnose, glucosamine or 2-deoxyglucose were used as the carbon source. However, in most studies only the qualitative analysis using GCMS of the resulting mixtures were published. The use of the Maillard reaction for large scale production of alkylpyrazines was mentioned only in some cases.

JP 2011062125 A describes a process for the production of pyrazine-flavored tallow using Maillard reaction between amino acids containing sodium L-aspartate and L-glutamic acid or a salt thereof and sugar. The process includes heating of a starting material in tallow at 180° C. for 10 minutes, filtration of solids and recovering the product. Also described is the use of thus produced flavored oil for aroma compositions.

The Maillard reaction products are complex and complicated because a large variety of alkylpyrazines can be obtained. In most cases unsubstituted pyrazine, methylpyrazine and dimethylpyrazine are the major products. The sensory properties of alkylpyrazines depend on the substitution pattern. In general, the odor threshold values decrease with increasing number of substituents within a homological series. In addition, ethylsubstituted pyrazines are usually more potent flavor compounds than methyl substituted pyrazines. For example, odor threshold values for methylpyrazine is 490 ppb, for trimethylpyrazine 38 ppb, for ethylpyrazine 57 ppb, for ethylmethylpyrazine 7 ppb, for diethylmethylpyrazine 4 ppb (Chemical Senses 1985, 10, 287-96). 2-Ethyl-3,5-dimethylpyrazine and 2,3-diethyl-5-methylpyrazine are two of the most potent naturally occurring alkylpyrazines, their odor threshold value is 4500-times less than that for trimethylpyrazine. (Zeitschrift für Lebensmittel-Untersuchung und-Forschung A: Food Research and Technology 999, 208, 308-316). Therefore, the generation of mixtures enriched in polysubstituted alkylpyrazines is of great relevance.

To date many chemical methods for generation of single alkylpyrazines have been published. The most important methods are summarized in this review (Borneo Journal of Resource Science and Technology 2017, 7, 60-75). However, these strategies require either special reaction conditions (such as very high temperatures, 300-600° C., or strictly anhydrous conditions), the use of metal catalysts or stoichiometric amounts of oxidation agents, or generate toxic waste, and thus, severely limit their scaling potential. Therefore, there is a high demand for a sustainable, environmentally friendly, cost-effective and selective approach towards the production of polysubstituted alkylpyrazines.

Selective formation of polysubstituted alkylpyrazines using hydroxyketones and/or dihydroxyketones instead of sugars for the Maillard reaction has been described (Agr Biol Chem 1990, 54, 1631-8; J. Agric. Food Chem 2008, 56, 2147-2153). EP 505891 A1 describes an approach for selective generation of dimethyl-, diethyl- or tetramethylpyrazines using a reaction between ammonium acetate and hydroxyacetone or hydroxybutanone. Furthermore, asymmetric substituted alkylpyrazines have been generated by using two different hydroxyketones. For example, starting from dihydroxyacetone and hydroxypentanone 2-ethyl-3,5-dimethylpyrazine and 3-ethyl-2,5-dimethylpyrazine have been generated (DE 69518206T2, EP 0708759 A1). However, hydroxyketones and dihydroxyketones are relatively instable and expensive and, hence, their use is not favorable for production at large scale.

Furthermore, the formation of alkylpyrazines through thermal decomposition of hydroxyamino acids without addition of any sugar was published. Serine and threonine, either alone or combined, were heated at 120° C. for 4 h or at 300° C. for 7 minutes. In the resulting mixtures traces of alkylsubstituted pyrazines were detected (120° C.: 125 ppm, 300° C.: 8040 ppm). Using serine, methylpyrazine, ethylpyrazine, 2-ethyl-6-methylpyrazine, 2,6-diethylpyrazine and unsubstituted pyrazine as the main compound were detected. Using threonine, 2,5-dimethylpyrazine, 2,6-dimethylpyrazine, trimethylpyrazine, 2-ethyl-3,6-dimethylpyrazine and 2-ethyl-3,5-dimethylpyrazine were generated. In this case, 2,5-dimethylpyrazine was identified as the main product (J. Agric. Food Chem 1999, 47, 4332-4335). A more detailed investigation of thermal decomposition of serine identified also the formation of several pyrrole derivatives (Food Chem. 2001, 74, 1-9). Addition of potassium carbonate was reported to accelerate the decomposition and change the selectivity of the reaction towards the formation of 3-ethyl-2,5-dimethylpyrazine as the main compound. However, no information about the isolated yields was published (Food Chem. 2009, 115, 1417-1423). Nothing is known with regard to possible large scale production capabilities of this strategy. Besides, the formation of alkylpyridines through thermal decomposition of hydroxyaminoacids was not mentioned.

There is a continuous demand for new authentic volatile flavor compounds. Especially naturally occurring flavors, which can be produced by green environmentally friendly methods, utilizing easily available, renewable raw materials and reactants, are of great interest. That is the reason why there is a continuing need for sustainable and cost-effective approaches towards polysubstituted alkylpyrazines such as ethyldimethyl- or diethylmethylpyrazine.

It was thus the primary object of the present invention to provide a process for producing alkylpyrazines that at least partially overcomes one or more of the above disadvantages.

The primary object of the present invention is achieved by a process as defined in appended claim 1, a process as defined in appended claim 13, an aromatic blend as defined in appended claim 14 and a composition as defined in appended claim 15.

Further aspects of the present invention or in connection therewith as well as preferred embodiments will be described below as well as in the attached claims.

### DETAILED DESCRIPTION OF THE INVENTION

The present invention primarily relates to a process for producing alkylpyrazines, the process comprising the following steps:
(a) thermal treatment of an amino acid source in a high boiling solvent, the amino acid source containing at least threonine and/or serine and the thermal treatment yielding alkylpyrazines;
(b) separation of the alkylpyrazines from the high boiling solvent and non-volatile sideproducts.

Surprisingly it was found that thermal treatment of an amino acid source containing threonine and/or serine in a high-boiling solvent provides a mixture of alkylpyrazines, comprising mainly asymmetric tri- and tetra-substituted alkylpyrazines. Surprisingly it was found that flavor blends thus prepared and compositions including thus prepared flavor blends exhibit a richer, more balanced and authentic flavor profile compared to single alkylpyrazines.

The process according to invention does not require unusual procedure steps such as very high temperature or strictly anhydrous conditions, so that the process can be easily realized in large scale without any problems. Furthermore, the process according to invention does not require expensive, mostly chemically produced and reactive hydroxyketones but employs a “green” starting material that is for instance producible by fermentation.

An amino acid source as used herein denotes a material containing or being constituted of amino acids, and includes without limitation sources of (single) amino acids, proteins, mixtures of amino acids and proteins as well as mixtures hydrolysates thereof. The phrase “an amino acid source containing at least threonine and/or serine” thus refers to a source in which threonine and/or serine is present in the form of (single) amino acids, in the form of amino acids being assembled in a protein chain or in the form of both. The description does not differentiate between peptides and proteins. Hence, the term “protein” encompasses a chain of two or more amino acids linked by peptide bond(s).

The term “thermal treatment” refers to a treatment at a temperature sufficient for decomposition of threonine and/or serine and formation of alkylpyrazines to occur resulting in thermal decomposition of the threonine and/or serine.

A high boiling solvent as used herein refers to a solvent having a boiling point of at least 200° C., preferably at least 225, most preferably 250° C. Particularly when the reaction is carried out at a temperature higher than 190° C., the boiling point is preferably at least 250° C. in order to avoid co-distillation of the solvent. It is to be understood that the high boiling solvent is stable under the reaction conditions including temperature and pH value. Moreover, the high boiling solvent does not participate in the reaction and is inert against potentially reactive intermediates (e.g. aldehydes, amines) formed during the thermal treatment.

Further aspects of the present invention or in connection therewith as well as preferred embodiments will be described below as well as in the attached claims.

A preferred method according to the present invention uses distillation and/or steam distillation to separate the alkylpyrazines from the high boiling solvent and non-volatile sideproducts. Non-volatile sideproducts are products other than the desired alkylpyrazines and alkylpyridines described herein and having a boiling point above 150° C., preferably above 200° C., more preferably above 250° C. Examples of non-volatile sideproducts include long chain fatty acids and amides thereof as well as polymeric reaction products formed from reactive aldehydes.

In certain embodiments, the distilled fraction contains water in addition to the alkylpyrazines. This is in particular so, if water is added prior to distillation to carry out steam distillation. In this case, it is preferred that the alkylpyrazines are subsequently extracted from the water containing distilled fraction. Water could generally also be present in the distilled fraction due to its formation during thermal treatment and/or due to its presence in the amino acid source in step (a) and/or due to its addition to the amino acid source in step (a). However, as described below, the water occurring and/or present in step (a) is preferably steadily distillated off.

Preferably, the separation of the alkylpyrazines in step b) is carried out at a temperature ranging from 90° C. to 150° C., preferably 100° C. to 130° C., and/or at a pressure of 0.5 to kPa, preferably at 1 to 5 kPa, using distillation. When using steam distillation, the separation of the alkylpyrazines in step b) is carried out at a temperature ranging from 100° C. to 140° C. and/or under ambient pressure (80 kPa to 120 kPa).

As mentioned above, the alkylpyrazines can be co-distillated with water, formed in step a). After thermal treatment, the reaction mixture can be diluted with water and distillated again to increase the isolated yield. Preferably, the reaction mixture is diluted in 1 to 3 proportions for this purpose. After distillation, the recovered mixture may contain up to 95 weight-% water. The separation of the alkylpyrazines from water is preferably carried out by phase separation and/or extraction. Using a combination of separation and extraction, the phases are separated first and subsequently the water phase is extracted, e.g. two times using an organic solvent. Particularly preferred is an extraction with a low-boiling solvent such as ethyl acetate or methyl tert-butyl ether, preferably ethyl acetate, followed by removal of solvent.

The resulting alkylpyrazines can then be distillated, in order to enrich specific alkylpyrazines, or directly used in aroma compositions. The distillation in this step aims at changing the amount of some alkylpyrazines relative to other alkylpyrazines according to the boiling point of the alkylpyrazines. Thereby, it is possible to further tune the sensory profile.

A preferred method as disclosed herein involves single amino acids as the principal component in the amino acid source. The term “principal component” as used herein denotes a component that is present in an amount of more than 50 weight-%, relative to the total weight of the amino acids present in the amino acid source. This means that, according to this preferred embodiment, more than half of the amino acids contained in or constituting the amino acid source are present as single amino acids and less than half thereof are assembled in protein(s) (amounts based on weight). Preferably, the amount of single amino acids in the amino acid source is at least 60 weight-%, preferably at least 70 weight-%, more preferably at least 80 weight-%, yet more preferably at least 90 weight-%, most preferably at least 95 weight-%, relative to the total weight of the amino acids present in the amino acid source.

A preferred amino acid source contains threonine and/or serine as the principal amino acid(s). In other terms, threonine and/or serine is (are) the most abundant amino acid(s) in the amino acid source (based on weight). A further preferred amino acid source contains threonine and/or serine in an amount of at least 20 weight-%, preferably at least 25 weight-%, more preferably at least 30 weight-%, yet more preferably at least 35 weight-%, yet more preferably at least 40 weight-%, yet more preferably at least 45 weight-%, yet more preferably at least 50 weight-%, yet more preferably at least 60 weight-%, yet more preferably at least 70 weight-%, yet more preferably at least 80 weight-%, most preferably at least 90 weight-%, relative to the total weight of the amino acids present in the amino acid source. When the amino acid source contains at least 20 weight-% threonine and/or serine, good yields of tri- and tetra-substituted alkylpyrazines can be achieved.

A further preferred amino acid source contains threonine as the principal amino acid. Preferably, threonine is present in an amount of at least 20 weight-%, preferably at least weight-%, more preferably at least 30 weight-%, yet more preferably at least 35 weight-%, yet more preferably at least 40 weight-%, yet more preferably at least 45 weight-%, yet more preferably at least 50 weight-%, yet more preferably at least 60 weight-%, yet more preferably at least 70 weight-%, yet more preferably at least 80 weight-%, most preferably at least 90 weight-%, relative to the total weight of the amino acids present in the amino acid source. The weight ratio of threonine to serine in a yet further preferred amino acid source is 1:5 to 5:1, preferably 1:4 to 4:1, more preferably 1:3 to 3:1, yet more preferably 1:2 to 2:1, most preferably 1:1.5 to 1.5:1. It is noted that pyrazines obtained from an amino acid source containing an 1:1 weight ratio of threonine to serine are different than but similarly preferred as pyrazines obtained from threonine alone. When the amino acid source contains threonine as the principal amino acid and/or threonine in excess to serine, a particularly advantageous mixture of alkylpyrazines and alkylpyridines can be generated.

According to a further embodiment of the invention the thermal treatment in step a) is carried out at a temperature ranging from 120° C. to 260° C., preferably 130° C. to 250° C., more preferably 140° C. to 240° C., yet more preferably 145° C. to 230° C., most preferably 150° C. to 220° C., and/or for a reaction time ranging from 1 to 30 h, preferably 2.5 to 28 h, more preferably 4 to 26 h, most preferably from 5 to 25 h. As will be apparent to the skilled person, the required reaction time depends on the temperature.

According to a further preferred embodiment, the thermal treatment in step (a) is carried out at a pressure of at least 200 kPa, preferably at least 300 kPa, more preferably at least 400 kPa, yet more preferably at least 500 kPa, yet more preferably at least 600 kPa, yet more preferably at least 700 kPa, yet more preferably at least 800 kPa, yet more preferably at least 900 kPa, most preferably at least 1000 kPa. A convenient way of doing so is to carry out the thermal treatment in a pressure-sealed container, such as a closed autoclave. For this purpose, the reaction mixture, i.e. the amino acid source along with further reactants, if present, is filled into and pressure-sealed by the container before the thermal treatment occurs. The pressure within the container will then rise as a result of the temperature increase during thermal treatment.

A preferred thermal treatment is carried out in the presence of a base. Preferably, the thermal treatment is carried out in the presence of 0.05 to 3.00 equivalent (eq), preferably to 2.5 eq, more preferably 0.07 to 2.0 eq, yet more preferably 0.08 to 1.5 eq, yet more preferably 0.09 to 1.2 eq, most preferably 0.1 to 1.0 eq base. The eq as described herein refers to the total weight of the amino acids present in the amino acid source subjected to the thermal treatment of step a). Preferably, the base is an organic base. It is further preferred that the base is selected from the group consisting of sodium, potassium, ammonium or calcium carbonates, hydrogen carbonates, acetates, formates, citrates and tartrates. Especially preferred are sodium acetate or sodium formate. The presence of a base allows the decomposition reaction to be accelerated and/or to occur at a lower temperature than in the absence of the base. Lowering the temperature provides the advantage that fewer undesired side products are formed. Particularly good results in this regard are obtained when the thermal treatment is conducted at 170° C. to 180° C. for about 7 h. A further advantage associated with a base is that the reaction mixture can be stirred more efficiently.

According to a preferred embodiment, the high-boiling solvent is an oil, preferably a vegetable oil. The vegetable oil is preferably selected from the group consisting of palm oil, palm seed oil, babasuu or cusi oil, hazelnut oil, coconut oil, sunflower oil, peanut oil, soya oil, raps oil and olive oil. Due to their broad applicability in food system, vegetable oils are especially suitable solvents for the transformation in step a) compared to other organic high boiling solvents. Further, vegetable oils are preferred over animal-based oils in view of the customer's and consumer's perspective.

In a preferred embodiment, the oil, preferably the vegetable oil as mentioned above, has a high content of saturated fatty acids (preferably≥50%, more preferably≥60%, most preferably≥70%,) like palm oil, palm seed oil, coconut oil and babasuu or cusi oil. This embodiment safeguards the oil to withstand and remain stable at the temperatures during thermal treatment.

Another embodiment prefers hazelnut oil as the high-boiling solvent for having a unique nutty flavor profile by its own.

It is to be understood that the high-boiling solvent can be a mixture of high-boiling solvents including or consisting of one or more of the above oils.

In a further preferred embodiment, the weight ratio of the amino acids in the amino acid source (whether present as single amino acids or in a protein chain) to the high-boiling solvent ranges from 10:1 to 1:10, preferably 8:1 to 1:8, more preferably 6:1 to 1:6, most preferably 4:1 to 1:4.

According to a preferred embodiment of the method according to invention, the amino acid source consists of serine and threonine. This embodiment results in the formation of tri- and tetra-substituted alkylpyrazines. Preferably, the formed alkylpyrazines contain at least 45% of tri- and tetra-substituted alkylpyrazines. The tri- and tetra-substituted alkylpyrazines may comprise:
2,5-dimethyl-3-ethylpyrazine and 3,5-dimethyl-2-ethylpyrazine (preferably 20 weight-% to 50 weight-%, more preferably 30 weight-% to 40 weight-%); and/or
2,3-diethyl-5-methylpyrazine and 2,5-diethyl-3-methylpyrazine (preferably 2 weight-% to weight-20%, more preferably 5 weight-% to weight-15%);
2,6-diethyl-3,5-dimethylpyrazine and 2,5-diethyl-3,6-dimethylpyrazine (preferably 2 weight-% to 15 weight-%, more preferably 5 weight-% to 10 weight-%).

The formed alkylpyrazines may further contain ethylmethyl pyrazines (preferably 5 weight-% to 20 weight-%, more preferably 5 weight-% to 15 weight-%).

According to a further preferred embodiment of the method according to invention, the amino acid source consists of threonine. This embodiment results in the formation of alkylpyrazines and alkylpyridines. Preferably, the formed mixture contains 20 weight-% to 60 weight-% of alkylpyrazines and alkylpyridines. The weight ratio of alkylpyrazines to alkylpyridines may be 5:1 to 1:5, preferably 2:1 to 1:2. Up to 70 weight-%, preferably up to weight-% of the alkylpyrazines can be composed of dimethylethyl pyrazines and/or diethyldimethyl pyrazines, such as 2,5-dimethyl-3-ethylpyrazine, 3,5-dimethyl-2-ethylpyrazine, 2,6-diethyl-3,5-dimethylpyrazine and/or 2,5-diethyl-3,6-dimethyl-pyrazine. Moreover, the weight ratio of the dimethylethyl pyrazines to the diethyldimethyl pyrazines is preferably 5:1 to 1:2, more preferably 3:1 to 1:1. It is further preferred that 5-ethyl-2-methylpyridine and 2,6-dimethyl-3-ethylpyridine are the main alkylpyridines formed (preferably up to 60 weight-%, more preferably up to 90 weight-%). The weight ratio of the to the 2,6-dimethyl-3-ethylpyridine is preferably 10:1 to 1:4, more preferably 4:1 to 1:1.

According to a further preferred embodiment of the method according to invention, the amino acid source consists of serine. This embodiment results in the formation of diethylpyrazine and ethylpyrazine. Preferably the formed alkylpyrazines contain diethylpyrazine to about 15 weight-% to 35 weight-%, preferably 20 weight-% to 30 weight-% and/or ethylpyrazine to about 10 weight-% to 35 weight-%, preferably 15 weight-% to 25 weight-%. In addition, the method may result in the formation of pyrrol derivatives (for instance up to 40 weight-%) in addition to the alkylpyrazines.

The present invention also relates to a process for producing a preparation for nourishment or pleasure, comprising carrying out the process as described herein and combining the alkylpyrazines with other ingredients of the preparation.

A further aspect of the present invention pertains to an aromatic blend containing one or more alkylpyrazines obtained or obtainable by the process of the invention. The aromatic blend is particularly advantageous as it can be obtained in an environmentally friendly manner from renewable sources without the use of petrochemical reagents. A preferred natural organic solvent used in the process of the invention is ethyl acetate, which can be recycled and reused.

Moreover, as described above, alkylpyrazines are valuable aroma compounds having roasted, nutty, chocolate flavor and taste, whereby a mixture of alkylpyrazines as described herein, comprising mainly asymmetric tri- and tetra-substituted alkylpyrazines, exhibit an even richer, more balanced and authentic flavor profile compared to single alkylpyrazines.

As described above, different combinations and ratios of alkylpyrazines and alkylpyridines can be obtained in the method described herein depending on the amino acid source. Surprisingly, it was found that, even at low concentrations of about 0.01 ppm, the obtained one or more alkylpyrazines (and optionally one or more alkylpyridines) can greatly intensify the nutty, especially hazel nutty, roasted, chocolate flavor and taste in orally consumable preparations. Thus, the different embodiments and features described in the context of the process of the invention form corresponding embodiments and features of the aromatic blend of the invention, in particular in regard of the combinations and ratios of alkylpyrazines and alkylpyridines formed. Moreover, aromatic blends are preferred, wherein the one or more alkylpyrazines are present in an amount between 0.01 ppm and/or below 0.3 ppm.

Preferably, the alkylpyrazines (and optionally the alkylpyridines) formed by the process of the invention are used without further manipulation of the amount and/or weight ratio of the formed alkylpyrazines (and optionally the alkylpyridines). This means that the desired amount and the desired ratio of the alkylpyrazines (and optionally the alkylpyridines) to be used in the aromatic blend of the invention is preferably set by the nature and the amount of starting substances, optionally in combination with the reaction conditions such as pH value, temperature and/or pressure but preferably not by adding or subtracting formed product(s). The aromatic blend thus prepared is particularly advantageous as it has a complex and authentic flavor and taste profile due to the presence of a high amount of different tri- and tetra-substituted alkylpyrazines.

According to a further aspect, the present invention relates to a composition, preferably a composition serving for food or pleasure, or a semi-finished product for producing said composition, comprising an aromatic blend as described herein, preferably in an amount sufficient for imparting, modifying and/or enhancing one or several flavor profiles selected from the group consisting of nutty, particularly hazelnut, roasted and chocolate. For this purpose, it is further preferred that the composition includes the aroma blend in an amount of at least 0.01 weight-%, preferably at least 0.1 weight-%, relative to the total weight of the composition. Preferably, the composition includes the aroma blend in an amount of 2 weight-% or less, preferably 1 weight-% or less, relative to the total weight of the composition.

The composition according to the invention is preferably an orally consumable food preparation and/or an orally consumable food supplement. The semi-finished product is preferably a semi-finished product for the production of an orally consumable food preparation and/or an orally consumable food supplement.

The aromatic blend of the invention or the composition of the invention, preferably a composition serving for food or pleasure, or a semi-finished product for producing said composition, may comprise one or more further, preferably volatile, aromatic substances. The further aromatic substance(s) may be used in the form of reaction flavors (Maillard-products), extracts or respectively, essential oils of plants or plant parts or, respectively, fractions thereof, smoke flavors or other flavor providing compositions (e.g. protein hydrolysates), grill-like flavors, plant extracts, spices, spice compositions, vegetables types and/or vegetable compositions. Particularly preferred are aromatic substances or their components that cause a roasted, nutty, sweet aromatic impression.

Furthermore, the aromatic blend of the invention or the composition of the invention, preferably a composition serving for food or pleasure, or a semi-finished product for producing said composition, may comprise one or more further ingredients selected from the group consisting of volatile organic acids, alcohols, thiols, disulfides, heterocyclic compounds (particularly pyrrolines, thiazols and thiazolines), aldehydes, ketones, esters and lactones. Specific examples in the respective groups include:
Organic acids: acetic acid, butyric acid, 2- or, respectively 3-methyl butyric acid, caprinic acid, capronic acid, phenylacetic acid;
Alcohols: ethanol, propylene glycol, 1,3-octenol, cis-3-hexenol, linalool, benzyl alcohol, p-cressol, 2,6-dimethylthiophenol, guajacol, eugenol;
Sulfides/thiols: dimethyl sulfide, difurfuryl disulfide, methyl thiopropanal, 2-methyl-3-methyldithiofuran and bis(2-methyl-3-furyl)disulfide, methylfuranthiol, 2-(4-methyl-1,3-thiazol-5-yl)ethanol (sulfurol), methyltetrahydrofuranthiol, 3-methyl-2-buten-1-thiol, 3-thio-2-methylpentanol, 2-furfurylthiol, thiophenol, 2-methylthiophenol and 2-mercaptobutanone;
Pyridines: 2-acetylpyridin;
Thiazoles/Thiazoline: 2-acetylthiazol, 2-acetyl-2-thiazolin;
Pyrrolines: 2-propionyl-1-pyrrolin and 2-acetyl-1-pyrrolin;
Aldehydes: acetaldehyde, trans-4,5-epoxy-(2E)-decenal, cis-4,5-epoxy-(2E)-decenal, (E,E)-2,4-undecadienal, (E,E)-2,4-decadienal, (E,Z)-2,4-decadienal, (E,E,Z)-2,4,6-nonatrienal, (E,E)-2,4-nonadienal, (E)-2-undecenal, (Z)-2-decenal, (E)-2-decenal, (E)-2-nonenal, (Z)-2-nonenal, (E,Z)-2,6-nonadienal, (E,E)-2,4-nonadienal, 3-methylthiopropanal (methional), vanillin and phenylacetaldehyd;
Ketones: 3,4-dimethylcyclopentan-1,2-dione, 3-hydroxy-4,5-dimethylfuran-2(5H)-on (sotolon), 2-aminoacetophenone, 3-hydroxy-4,5-dimethyl-2(5H)-furanone, 2,5-dimethyl-4-hydroxy-3-[2H]-furanone (furaneol©), tetrahydrothiophen-3-one and 3-thiobutan-2-one; Esters and lactones: methylbutanoate, ethyl-3-methylbutanoate, propyl-2-methylbutanoate, (Z)-6-dodecen-y-lactone, 4-hydroxy-2-nonenoic acid lactone, δ-undecalactone, γ-nonalactone, dodecalactone and γ-octalactone.

Compositions serving for food or pleasure in particular include: baked goods (e.g. bread, dry cookies, cake, other baked goods), sweets (e.g. chocolates, chocolate bar products, other bar products, fruit gum, hard and soft caramels, chewing gums), alcoholic or non-alcoholic drinks (e.g. cocoa, coffee, green tea, black tea, (black, green) tea drinks enriched with (green, black) tea extracts, rooibos-tea, other herbal teas, wine, drinks containing wine, beer, drinks containing beer, liqueurs, schnapps, brandies, lemonades containing fruits, isotonic drinks, refreshing-drinks, nectars, fruit and vegetable juices, fruit or vegetable juice preparations), instant drinks (e.g. instant cocoa drinks, instant tea drinks, instant coffee drinks), meat products (e.g. ham, fresh sausage or raw sausage compositions, spiced or marinated fresh or salt meat products), eggs or egg products (dry egg, protein, yolk), wheat products (e.g. breakfast cereals, muesli bars, precooked finished-rice products), dairy products (e.g. full fat or fat-reduced or fat-free milk drinks, rice pudding, yoghurt, pudding, kefir, cream cheese, soft cheese, hard cheese, dry milk powder, whey, butte, buttermilk, partly or completely hydrolyzed milk protein containing products), products made of soy protein or other soy bean fractions (e.g. soy milk and products made thereof, isolated or enzymatically treated soy protein containing drinks, soy flour containing drinks, soya lecithin containing compositions, fermented products such as tofu or tempe or products made thereof and mixtures with fruit compositions and facultative fragrances), fruit compositions (e.g. jams, sorbets, fruit sauces, fruit fillings), vegetable compositions (e.g. ketchup, sauces, dry vegetables, frozen vegetables, pre-cooked vegetables, boiled down vegetables), snacks (e.g. baked or fried potato chips or potato dough products, extrudates based on corn or peanut), products based on fat and oil or emulsions of the same (e.g. mayonnaise, remoulade, dressings, each full fat or fat-reduced), other finished-products and soups (e.g. dry soups, instant soups, pre-cooked soups), spices, spice compositions as well as particularly seasonings, which are e.g. used in the field of snacks, sweetener compositions, sweetener tablets or sweetener sachets, other compositions for sweetening or whitening of drinks or other food.

The composition as described herein may further include one or more (typical) basic materials, excipients or additives for foodstuff or luxury food.

Examples for typical basic materials, excipients or additives include water, mixtures of fresh or processed, vegetable or animal basic or raw materials (e.g. raw, roasted, dried, fermented, smoked and/or cooked meat, bone, cartilage, fish, fish, vegetable, fruits, herbs, nuts, vegetable or fruit juices or pastes or their mixtures), digestible or non-digestible carbohydrates (e.g. saccharose, maltose, fructose, glucose, dextrins, amylose, amylopectin, inulin, xylene, cellulose), sugar alcohols (e.g. sorbate), natural or hardened fats (e.g. sebum, lard, palm fat, coconut oil, corn oil, olive, fish oil, soy oil, sesame oil).

The one or more basic materials, excipients or additives may be present in amounts of from 5 to 99.999999 weight-%, preferably 10 to 80 weight-%, relative to the total weight of the composition. Water may be present in an amount of up to 99.999999 weight-%, preferably 5 to 80 weight-%, relative to the total weight of the composition.

The composition as described herein may further include one or more of the following (preferably in an amount of 10 to 5000 ppm, preferably 50 to 1000 ppm, relative to the total weight of the composition): fatty acid or their salts (e.g. potassium stearate), proteinogenic or non-proteinogenic amino acids and related compounds (e.g. taurine), peptides, native or processed proteins (e.g. gelatin), enzymes (e.g. peptidases), nucleic acids, nucleotides, taste correctants for unpleasant taste impressions (e.g. hesperetin, phloretin or other hydroxychalcon derivatives to be used according to US 2008/0227867 as well as optionally the lactones mentioned there), taste modulating substances (e.g. inositol phosphate, nucletides such as guanosine monophosphate, adenosine monophosphate or other substances such as sodium glutamate or 2-phenoxy propionic acid), emulsifiers (e.g. lecithins, diacylglycerols), stabilizers (e.g. carrageenan, alginate), preservatives (e.g. benzoic acid, sorbic acid), antioxidants (e.g. tocopherol, ascorbic acid), chelators (e.g. citric acid), organic or inorganic acidifiers (e.g. malic acid, acetic acid, citric acid, tartaric acid, phosphoric acid, lactic acid), additional bitter substances (e.g, chinine, caffeine, limonin, amarogentin, humolone, lupolone, catechins, tannins), sweeteners (e.g. saccharin, cyclamate, aspartame, neotame, steviosides, rebaudiosides, acesulfam K, neohesperidin hydrochalcone, thaumatin, superaspartame), mineral salts (e.g. sodium chloride, potassium chloride, magnesium chloride, sodium phosphate), substances inhibiting the enzymatic browning (e.g. sulfite, ascorbic acid), essential oils, plant extracts, natural or synthetic dyes or dye pigments (e.g. carotinoids, flavonoids, anthocyans, chlorophyll and their derivatives) spices, synthetic, natural or nature identical aromatic substances or flavors such as olfactory correctants.

All of the ingredients/substances/components disclosed herein are meant to be combinable with all other ingredients/substances/components disclosed herein.

The aromatic blend or composition as described herein preferably contains the alkylpyrazines (obtained or obtainable by the method as described herein) as pure material, i.e. without any solvent, or as a solution, i.e. including a solvent. Preferred are 0.1 to 20 weight-%, particularly preferred 1 to 10 weight-%, solutions in triacetine. In addition, liquid compositions can be spray dried to get solid flavors.

The present invention is subsequently further explained by means of the following examples. The examples have the purpose of clarifying the invention and are not intended to limit the scope of the claims. Unless stated otherwise, all amounts refer to weight.

### EXAMPLES

### Example 1

To 240 g of serine were added 240 g of threonine and 93.7 g of sodium acetate and 480 g of coconut oil. The resulting reaction mixture was then heated for 7 h at 175° C. while stirring. The resulting water was steadily distillated off. After 7 h the reaction mixture was cooled to 80° C. and 200 mL water were added. The product was distilled off together with water at 120° C. and normal pressure. The obtained distillate was extracted with 400 mL ethyl acetate. The organic phase was finally concentrated under reduced pressure (70 mbar, 30° C.) to give 7.27 g of the following alkylpyrazines.

GC Analysis (FID, without standard, DB-1, 60-9-240°): 28.3% 2,5-dimethyl-3-ethylpyrazine, 10.8% 2-ethyl-5(6)-methylpyrazine, 9.4% 3,5-dimethyl-2-ethylpyrazine, 9.1% trimethyl-pyrazine and 2-ethyl-3-methylpyrazine, 8.3% 2,6-dimethylpyrazine, 7.3% 2,3-diethyl-5-methylpyrazine, 4.8% 2,6-diethyl-3,5-dimethylpyrazine, 3.0% 2-ethyl-3,5,6-trimethylpyrazine, 2.8% 2,5-diethyl-3-methylpyrazine, 2.2% methylpyrazine, 1.1% 2,5-diethyl-3,6-dimethylpyrazine, 0.7% ethyl-methyl-propylpyrazine, 0.7% ethylpyrazine.

### Example 2

To 120 g of serine were added 23.4 g of sodium acetate and 120 g of coconut oil. The resulting reaction mixture was then heated for 7 h at 175° C. while stirring. The resulting water was steadily distillated off. After 7 h the reaction mixture was cooled to 80° C. and mL water were added. The product was distilled off together with water at 120° C. and normal pressure. The obtained distillate was extracted with 200 mL ethyl acetate. The organic phase was finally concentrated under reduced pressure (70 mbar, 30° C.) to give g of the following alkylpyrazines.

GC Analysis (FID, without standard, DB-1, 60-9-240°): 21% 2.6-diethylpyrazine, 17.7% pyrrol, 15.6% ethylpyrazine, 12.0% N-(2-hydroxyethyl)-pyrrol, 4.2% 2,3-dimethyl-5-ethylpyrazine, 3.1% 2-ethyl-5-methylpyrazine, 3% 2,3-diethyl-5,6-dimethylpyrazine, 2.1% 2-ethyl-3-methylpyrazine, 1.6% 2,5-diethyl-pyrazine, 1.1% 2-ethyl-3,5,6-trimethylpyrazine, 0.7% methylpyrazine, 0.4% 5-isopropyl-2,3-dimethyl-pyrazine.

### Example 3

To 120 g of threonine were added 41 g of sodium acetate and 120 g of coconut oil. The resulting reaction mixture was then heated for 15 h at 175° C. while stirring. The resulting water was steadily distillated off. After 15 h the reaction mixture was cooled to 80° C. and 80 mL water were added. The product was distilled off together with water at 120° C. and normal pressure. The obtained distillate was extracted with 200 mL ethyl acetate. The organic phase was finally concentrated under reduced pressure (70 mbar, 30° C.) to give 1.6 g of the following alkylpyrazines.

GC Analysis (FID, without standard, DB-1 60-9-240°): 19.2% 2,5-dimethyl-3-ethylpyrazine, 12.2% 5-ethyl-2-methylpyridine, 5.7% 3,5-dimethyl-2-ethylpyrazine, 4.6% 2,5-diethyl-3,6-dimethyl-pyrazine, 3.7% 2,6-diethyl-3,5-dimethylpyrazine, 3.0% 2-ethyl-3,5,6-trimethylpyrazine, 2.4% 2,6-dimethyl-3-ethylpyrazine, 2.1% ethyl-methyl-propylpyrazine, 1.6% 2,6-dimethyl-3-ethylpyridine, 1.6% 2,5-diethyl-pyridine, 1.1% trimethylpyrazine, 1.1% 2,5-dimethylpyrazine, 0.7% 2,5-dimethylpyridine.

### Example 4

To 60 g of serine were added 60 g of threonine and 50 g of arginine and 120 g of coconut oil. The resulting reaction mixture was then heated for 7 h at 175° C. while stirring. The resulting water was steadily distillated off. After 7 h the reaction mixture was cooled to 80° C. and 80 mL water were added. The product was distilled off together with water at 120° C. and normal pressure. The obtained distillate was extracted with 200 mL ethyl acetate. The organic phase was finally concentrated under reduced pressure (70 mbar, 30° C.) to give 0.2 g of the following alkylpyrazines.

GC Analysis (FID, without standard, DB-1, 60-9-240°): 13.9% 2,5-dimethyl-3-ethylpyrazine, 11.2% 2-ethyl-3,5,6-trimethyl-pyrazine, 9.3% 2,5-diethyl-3,6-dimethylpyrazine, 7.3% 3,5-dimethyl-2-ethylpyrazine, 7.1% 2-ethyl-3-methylpyrazine, 6.0% 2,3-diethyl-5-methylpyrazine, 4.8% pyrrole, 4.0% 2-ethyl-5(6)-methyl-pyrazine and trimethylpyrazine, 2.7% triethylmethylpyrazine, 2.6% ethylpyrazine, 2.5% 2,3-diethylpyrazine, 1.7% 2,6-diethyl-3,5-dimethylpyrazine, 1.5% 2,6-dimethylpyrazine, 1.0% 2,3-dimethylpyrazine.

### Example 5

To 20 g of serine were added 20 g of threonine and 40 g of coconut oil. The resulting reaction mixture was then heated for 2 h at 185° C. while stirring in closed autoclave. After cooling to room temperature the reaction mixture was distillated at 140° C. and 3 mbar to give 0.7 g of the following alkylpyrazines.

GC Analysis (FID, without standard, DB-1, 60-9-240°): 31% 2,5-dimethyl-3-ethylpyrazine, 13.2% 2-ethyl-5(6)-methylpyrazine, 9.7% 2,6-dimethylpyrazine, 7.6% 3,5-dimethyl-2-ethylpyrazine, 6.8% trimethylpyrazine, 4.4% 2-ethyl-3,5,6-trimethylpyrazine, 3.1% 2-ethyl-3-methylpyrazine, 2.9% methylpyrazine, 2.7% 2,5-diethyl-3,6-dimethylpyrazine, 1.7% 2,3-diethyl-5-methylpyrazine, 1.7% 2,6-diethyl-3,5-dimethylpyrazine, 1.4% ethylpyrazine.

### Example 6

To 600 g of threonine were added 338 g of sodium acetate and 600 g of coconut oil. The resulting reaction mixture was then heated for 13 h at 175° C. while stirring. The resulting water was steadily distillated off. After 13 h the reaction mixture was cooled to 80° C. and 130 mL water were added. The product was distilled off together with water at 120° C. and normal pressure. The obtained distillate was extracted with 200 mL ethyl acetate. The organic phase was finally concentrated under reduced pressure (70 mbar, 30° C.) to give 8.5 g of the following alkylpyrazines.

GC Analysis (FID, without standard, DB-1, 60-9-240°): 11.6% 5-ethyl-2-methylpyridine, 5.9% 2,5-dimethyl-3-ethylpyrazine, 5.3% 2,6-dimethyl-3-ethylpyridine, 4.4% 2,6-diethyl-3,5-dimethylpyrazine, 4.4% ethyl-methyl-propylpyrazine, 3.1% 2,5-diethyl-3,6-dimethylpyrazine, 2.1% 2,5-diethylpyridine, 1.9% 3,5-dimethyl-2-ethylpyrazine, 1.7% 2-ethyl-3,5,6-trimethylpyrazine.

### Aroma Compositions

### Application Example 1

### Hazelnut Flavor

Hazelnut flavors were prepared by compounding the ingredients shown in the following table.

| # | Ingredients | A | B | C | D |
|---|---|---|---|---|---|
| 1 | Acetylpyrazin-2 | 2.5 g | 2.5 g | 2.5 g | 2.5 g |
| 2 | Vanillin | 25.0 g | 25.0 g | 25.0 g | 25.0 g |
| 3 | Tetrahydrochinoxaline | 3.0 g | 3.0 g | 3.0 g | 3.0 g |
| 4 | Benzaldehyde | 8.0 g | 8.0 g | 8.0 g | 8.0 g |
| 5 | Aldehyde C18 so called | 1.0 g | 1.0 g | 1.0 g | 1.0 g |
| 6 | Trimethylpyrazine | 1.0 g | 1.0 g | 1.0 g | 1.0 g |
| 7 | 3,4, 1,2- | 6.0 g | 6.0 g | 6.0 g | 6.0 g |
| | Dimethylcyclopentandione | | | | |
| 8 | 1,2-Propylenglykol | 950.5 g | 947.2 g | 947.3 g | 940.5 g |
| 9 | 2,3-Pentandione | 3.0 g | 3.0 g | 3.0 g | 3.0 g |
| 10 | Dimethylethylpyrazine- | — | 3.3 g | — | — |
| | 3,5(6),2 | | | | |
| 11 | Pyrazine mixture | — | — | 3.3 g | — |
| | from example 6 | | | | |
| | (10% in Triacetine) | | | | |
| 12 | Pyrazine mixture from | — | — | — | 10 g |
| | example 1 | | | | |
| | (10% in Triacetine) | | | | |

| | | | | |
|---|---|---|---|---|
| Total | 1000 g | 1000 g | 1000 g | 1000 g |

A, B = compositions of comparison;
C, D = compositions according to the invention

### Sensory Evaluation

50 g sugar and 0.3 g of hazelnut flavor were dissolved in 1000 mL water. When tested by a panel of skilled persons, the compositions according to the invention C and D are assessed as clearly more nutty especially more hazelnutty and more pyrazine-like with regard to its aroma and taste then the composition of comparison A and B.

### Application Example 2

### Chocolate Flavor

Seven chocolate flavors were prepared by compounding the ingredients shown in the following table.

| # | Ingredient | A | B |
|---|---|---|---|
| 1 | Acetylmethylcarbinol | 2 g | 2 g |
| 2 | Anisaldehyde 1% | 0.5 g | 0.5 g |
| 3 | Benzaldehyde 10% | 0.2 g | 0.2 g |
| 4 | Butyric acid | 0.05 g | 0.05 g |
| 5 | Cinnamic aldehyde | 0.1 g | 0.1 g |
| 6 | Cinnamylacetate | 1 g | 1 g |
| 7 | Para-Cresol 0, 1% | 0.2 g | 0.2 g |
| 8 | Decalactone delta | 0.2 g | 0.2 g |
| 9 | Dodecalactone delta | 0.1 g | 0.1 g |
| 10 | Ethylcaproate | 0.05 g | 0.05 g |
| 11 | Furaneol 15% | 5 g | 5 g |
| 12 | Guajacol 1% | 0.5 g | 0.5 g |
| 13 | Isoamylalkohol | 2 g | 2 g |
| 14 | Isobutyraldehyde | 0.5 g | 0.5 g |
| 15 | Isovaleric acid | 0.1 g | 0.1 g |
| 16 | Iso-valeric aldehyde | 2 g | 2 g |
| 17 | 3,2,2-Methylcyclopentenolone | 2 g | 2 g |
| 18 | Methylguajacol-4 | 0.1 g | 0.1 g |
| 19 | 5,2,2-Methylphenylhexenal | 2 g | 2 g |
| 20 | Phenylacetaldehyde | 1 g | 1 g |
| 21 | Phenylacetic acid | 2 g | 2 g |
| 22 | Ethylphenylacetate | 0.5 g | 0.5 g |
| 23 | Phenylethylalcohol | 0.5 g | 0.5 g |
| 24 | Vanille extract 3X | 5 g | 5 g |
| 25 | Vanillin | 50 g | 50 g |
| 26 | Cacao extract | 50 g | 50 g |
| 27 | Triacetine | 250 g | 250 g |
| 28 | Propylenglycol-1,2 | 621.25 g | 616.25 g |
| 29 | Pyrazine mixture from examples | — | 5 g |
| | 1 to 6 (10% in Triacetine) | | |

| | | |
|---|---|---|
| Total | 1000 g | 1000 g |

A = composition for comparison;
B = compositions according to the invention

Then, 80 g sugar and 0.5 g of chocolate flavor were dissolved in 1000 mL water.

When tested by a panel of skilled persons, the six compositions according to the invention B are assessed as clearly more rich and balanced and cacao notes are intensified with regard to its aroma and taste then the composition of comparison A.

### Application Example 3

### Chicken Flavor, Boiled Type

Seven chicken flavors were prepared by compounding the ingredients shown in the following table.

| # | Ingredients | A | B |
|---|---|---|---|
| 1 | Decadienal trans, trans-2,4 | 15 g | 15 g |
| 2 | Decalactone gamma | 1.5 g | 1.5 g |
| 4 | Decenal trans-2 | 5 g | 5 g |
| 5 | Furaneol 10% in triacetine | 150 g | 150 g |
| 6 | Methylcyclopentenolon-3,2,2 | 10 g | 10 g |
| 7 | Methylfuranthiol-2,3 5% TRIC. | 25 g | 25 g |
| 8 | Methylthiopropanal-3 | 1 g | 1 g |
| 9 | 1,3-Octenol | 0.5 g | 0.5 g |
| 10 | Vegetable oil triglyceride palm free | 792 g | 784.5 g |
| 11 | Pyrazine mixture from example | — | 7.5 g |
| | 1 to 6 (10% in Triacetine) | | |
| | Total | 1000 g | 1000 g |

A = composition for comparison;
B = composition according to the invention

Then, 0.1 g of chicken flavor were dissolved in 1000 mL of bouillon.

When tested by a panel of skilled test persons, the compositions according to the invention B are assessed as clearly more authentic and roasted notes are intensified with regard to its aroma and taste then the composition of comparison A.

### Application Example 4: Spray Dried Aroma Composition

Starting from the previously described application examples 1 to 3, spray dried aroma compositions are prepared as follows:

| # | Ingredient | A | B | C | D | E | F |
|---|---|---|---|---|---|---|---|
| 1 | Capsule | 200 g | 200 g | 200 g | 200 g | 200 g | 200 g |
| 2 | Maltodextrin | 600 g | 600 g | 600 g | 600 g | 600 g | 600 g |
| 3 | Aroma composition | 200 g | | — | | — | |
| | application example 1A | | | | | | |
| 4 | Aroma composition | — | 200 g | — | — | — | — |
| | application example 1C | | | | | | |
| | or 1D | | | | | | |
| 5 | Aroma composition | — | — | 200 g | — | — | — |
| | application example 2A | | | | | | |
| 6 | Aroma composition | — | — | — | 200 g | — | — |
| | application example 2B | | | | | | |
| 7 | Aroma composition | — | — | — | — | 200 g | — |
| | application example 3A | | | | | | |
| 8 | Aroma composition | — | — | — | — | — | 200 g |
| | application example 3B | | | | | | |
| 9 | Water | 1000 g | 1000 g | 1000 g | 1000 g | 1000 g | 1000 g |

The ingredients are dissolved in demineralized water and subsequently spray dried. The spray dried aroma compositions are used in the subsequent application examples.

### Application Example 5

### Hazelnut Pudding

Hazelnut puddings are prepared from the spray dried aroma compositions by mixing the following ingredients:

| # | Ingredient | A | B |
|---|---|---|---|
| 1 | Sucrose | 7.8 g | 7.8 g |
| 2 | Cacao | 1 g | 1 g |
| 3 | Starch | 3.0 g | 2.75 g |
| 4 | Skimmed milk powder | 1.5 g | 1.5 g |
| 5 | Aubygel MR50 | 0.5 g | 0.5 g |
| 6 | Extract of vanilla beans, spray dried | 0.1 g | 0.1 g |
| 7 | Spray dried aroma composition example 4A | 0.25 g | — |
| 8 | Spray dried aroma composition example 4B | — | 0.25 g |
| 9 | Milk 1.5% fat content | 100 g | 100 g |

A = composition for comparison;
B = composition according to the invention

The ingredients are dissolved in milk warmed to 95° C. for 2 minutes while stirring well, and subsequently cooled to 5 to 8° C.

It is shown that by using the pyrazine containing compositions of the invention, a clearly hazelnut, chocolate, authentic taste is achieved.

### Application Example 6

### Chocolate

Chocolates are prepared from the spray dried aroma compositions by mixing the following ingredients:

| # | Ingredient | A | B |
|---|---|---|---|
| 1 | Cacao butter | 14.49 g | 14.49 g |
| 2 | Cacao paste | 41.00 g | 41.00 g |
| 3 | Sucrose | 43.88 g | 43.88 g |
| 4 | Lecithin | 0.48 g | 0.48 g |
| 5 | Vanillin | 0.02 g | 0.02 g |
| 6 | Chocolate flavor according to example 2A | 0.1 g | — |
| 7 | Chocolate flavor according to example 2B | — | 0.1 g |
| | Total | 100 g | 100 g |

A = composition for comparison;
B = composition according to the invention

When tested by a panel of skilled test persons, the cacao notes of the chocolate according to the invention B are assessed more rich and balanced in comparison with composition A.

### Application Example 7

### Instant Soup, Type Chicken Soup with Noodles

Instant soups are prepared from the spray dried aroma compositions of application example 4 by mixing the following ingredients:

| # | Ingredient | A | B |
|---|---|---|---|
| 1 | Starch | 16 g | 16 g |
| 2 | Sodium chloride | 7 g | 7 g |
| 3 | Saccharose, refined | 3.2 g | 3.2 g |
| 4 | Sodium glutamate | 3.2 g | 3.2 g |
| 5 | Sodium inosinate/sodium | 0.8 g | 0.8 g |
| | guanylate in a ratio of 1:1 | | |
| 6 | acid hydrolyzed plant protein | 8.0 g | 8.0 g |
| 7 | Fat power | 2.0 g | 2.0 g |
| 8 | Vegetable fat, spray dried | 1.0 g | 1.0 g |
| 9 | Freeze-dried chicken meat, in pieces | 2.15 g | 2.15 g |
| 10 | Soup noodles | 32 g | 32 g |
| 11 | maltodextrin | 11.4 g | 11.4 g |
| 12 | Chinese vegetables, freeze-dried | 4.6 g | 4.6 g |
| 13 | Chicken flavor | 7.5 g | 7.5 g |
| 14 | Food dye riboflavin | 0.05 g | 0.05 g |
| 15 | Spray dried aroma compositions of | 1.1 g | — |
| | application example 4E | | |
| 16 | Spray dried aroma compositions of | — | 1.1 g |
| | application example 4F | | |
| | Total | 100 g | 100 g |

A = composition for comparison;
B = composition according to the invention

4.6 g of the mixed ingredients are boiled in 100 mL water for 10 minutes to obtain a ready-to-eat soup.

It is found that the composition according to the invention B results in clearly more rich, balanced, authentic and long-lasting profile.

1. A process for producing alkylpyrazines, the process comprising:
(a) thermally treating an amino acid source in a high boiling solvent, the amino acid source comprising at least threonine and/or serine, wherein the thermal treatment yields alkylpyrazines; and
(b) separating the alkylpyrazines from the high boiling solvent and non-volatile side-products, wherein the high boiling solvent is a solvent having a boiling point of at least 200° C.

2. Process of claim 1, wherein the alkylpyrazines are separated from the high boiling solvent and non-volatile side-products by distillation.

3. The process of claim 2, wherein separation of the alkylpyrazines is carried out at a temperature of 90° C. to 150° C.

4. The process of claim 2, wherein after distillation the alkylpyrazines are separated from water by extraction.

5. The process of claim 1, wherein the amino acid source comprises individual amino acids as a principal component.

6. The process of claim 1, wherein the amino acid source comprises threonine and/or serine as principal amino acid(s).

7. The process of claim 1, wherein the threonine in the amino acid source is in an amount of at least 20 weight-%, relative to a total weight of all amino acids present in the amino acid source, and/or wherein the threonine and the serine in the amino acid source are in a weight ratio of at least 1:1 (threonine:serine).

8. The process of claim 1, wherein the thermal treatment is carried out at a temperature of 120° C. to 260° C. and/or for a reaction time of 1 to 30 h and/or at a pressure of at least 200 kPa.

9. The process of claim 1, wherein the thermal treatment is carried out with an amount of a base.

10. The process of claim 1, wherein the high-boiling solvent is an oil.

11. The process of claim 1, wherein a weight ratio of the amino acids in the amino acid source to the high-boiling solvent is 10:1 to 1:10 (amino acids:high-boiling solvent).

12. The process of claim 1, wherein the amino acid source consists of:
(i) serine; or
(ii) serine and threonine; or
(iii) threonine.

13. A process for producing an edible preparation comprising:
(a) carrying out the process of claim 1; and
(b) combining the alkylpyrazines with other ingredients of the preparation.

14. An aromatic blend comprising alkylpyrazines obtained or obtainable by the process of claim 1.

15. A preparation comprising the aromatic blend of claim 14.

16. The process of claim 1, wherein the threonine in the amino acid source is in an amount of at least 20 weight-%, relative to a total weight of all amino acids present in the amino acid source, and the threonine and the serine in the amino acid source are in a weight ratio of at least 5:1 (threonine:serine).

17. The process of claim 1, wherein the thermal treatment is carried out at a temperature of 120° C. to 260° C., for a reaction time of 1 to 30 h, and at a pressure of at least 500 kPa.

18. The process of claim 1, wherein the thermal treatment is carried out with a base in an amount of 0.05 to 3.00 equivalent (eq).

19. The process of claim 1, wherein the high-boiling solvent is vegetable oil.

20. The process of claim 1 wherein a weight ratio of the amino acids in the amino acid source to the high-boiling solvent is 6:1 to 1:6 (amino acids:high-boiling solvent).
